(19) United States
(12) Patent Application Publication
(10) Pub. No.: US 2025/0037881 A1
(43) Pub. Date: Jan. 30, 2025

(54) SYSTEMS AND METHODS FOR AUTOMATED ASSESSMENT OF CLINICAL SUITABILTY OF MEDICAL DATA

(71) Applicants: Aerin Cox, Annandale, VA (US); Ali Moeeny, Belle Mead, NJ (US)

(72) Inventors: Aerin Cox, Annandale, VA (US); Ali Moeeny, Belle Mead, NJ (US)

(21) Appl. No.: 18/787,452

(22) Filed: Jul. 29, 2024

Related U.S. Application Data
(60) Provisional application No. 63/529,449, filed on Jul. 28, 2023.

(51) Int. Cl. G16H 50/70; G16H 10/60; G16H 30/40

(52) U.S. Cl. CPC G16H 50/70; G16H 10/60; G16H 30/40

(57) ABSTRACT

The invention provides systems and methods for providing automated assessment of suitability of medical data for clinical use, including use for the diagnosis of disease and/or planning or evaluation of treatment.

### CROSS-REFERENCE TO RELATED APPLICATIONS

This application claims priority to, and the benefit of, U.S. Provisional Application No. 63/529,449, filed on Jul. 28, 2023, the content of which is incorporated by reference herein in its entirety.

### TECHNICAL FIELD

The disclosure relates generally to medical data review, and, more particularly, to a system for providing automated assessment of suitability of medical data for clinical use, including use for the diagnosis of disease and/or planning or evaluation of treatment.

### BACKGROUND

Medical device manufacturers generally need to obtain approval from regulatory bodies (e.g. FDA and the like) in order to sell devices in the clinical market. Despite the fact that there are numerous pathology whole slide imaging (WSI) scanners in the non-clinical market, only a handful of WSI scanners are approved in the U.S. only to be used for a narrow subset of pathology subspecialties. In fact, there are no WSI scanners currently approved for blood slide imaging or for cytology, among many other pathology imaging tasks. It should be noted that digital microscopes, digital monitors, software for viewing pathology slides, as well as artificial intelligence (AI) pathology software solutions are facing similar deficits in regulatory approvals.

Some common reasons as to why so few pathology imaging devices are approved include, for example, the excessive amount of time, excessive amount of money, and inflexibility associated with attempting to gain approval from regulatory bodies.

For example, if a pathology imaging device manufacturer pursues regulatory approval, they need to run lengthy and costly trials to show parity between their device and the established standard of care (e.g. the use of analog optical microscopes). According to a 2010 survey published by Stanford University, a single approval can take a company 31-54 months for the FDA, and 7-11 months in Europe. Unfortunately, after receiving approval for a given device, the manufacturer cannot easily change anything about the hardware or software of the device. If any changes were made or elements were added to the device, the manufacturer would likely have to reapply for approval, thereby restarting the process and incurring additional time and costs. This is especially difficult for software and Artificial Intelligence (AI) companies who rely on frequent updates and iterations of their software.

Furthermore, if a medical device uses images as input, the manufacturer must specify the particular hardware that produces the image. If the manufacturer or the clinical users of the device desire to use any other hardware's images, or change the hardware, reapproval must be pursued. As such, for software companies that wish to work with many different hardware models, this is practically an insurmountable burden.

### SUMMARY

The invention of the present disclosure addresses issues currently faced by medical device manufacturers with regard to regulatory approval. In particular, the present invention is directed to a system for providing automated assessment of the suitability of medical data produced by hardware or software medical devices for their use in patient care. Such medical data (also referred to as healthcare data) may include, but is not limited to, digital data that may be in the form of image data generally associated with a patient's medical examinations or tests.

More specifically, the present invention includes a system for evaluating the suitability of medical images for clinical use, including diagnosis of disease, as well as planning and/or evaluation of treatments. In some embodiments, the medical images may be associated with histologic examinations, which may include one or more images of patient tissue specimens that may be collected and prepared via histological-related techniques. For example, the system is configured to receive one or more images of a histologic examination (i.e., images of one or more histologically prepared tissue samples), such as a peripheral blood smear digitized by a slide scanner. However, it should be noted that the medical images may be associated with certain examinations/tests generally performed via a medical imaging modality and thus may include medical images provided via x-ray imaging, Magnetic Resonance Imaging (MRI), Computed Tomography (CT) imaging, ultrasound (US) imaging, and the like. It should further be noted that, in some embodiments, the medical data may include other forms of data associated with medical- or healthcare-related examinations and tests.

The system is further configured to receive a request from a user associated with a clinical task or use case, essentially requesting that the system determine whether the one or more images are suitable for use in accomplishing such a requested clinical task. For example, a user may request that the system assess whether the one or more images are suitable for use in the clinical task of detection of sickle cells in a patient's blood.

In turn, the system is configured to analyze the one or more images, including evaluating the one or more images in different stages and at different predefined levels using a variety of evaluation techniques to determine the suitability of the one or more images for being used for the requested clinical task.

For example, the system is configured to process the request data (i.e., the one or more medical images and the user's clinical task request) so as to provide a validation of the request data (i.e., validating various parameters of the inputted medical image data for the purposes of performing the clinical task requested). Such validation of the request data includes, but is not limited to: 1) validation of the integrity of the medical image (i.e., to ensure that medical image data has not been modified, degraded, or compromised during transmission to the system or during storage thereof); 2) validation of the of the user's request (i.e., verifying that the specifical clinical task request by the user is available for processing by the system); 3) validation of accuracy of metadata associated with the medical image data (i.e., to ensure that the received medical image data matches metadata information); 4) validation of physical characteristics of the medical image (i.e., to ensure that certain physical characteristics, such as optical focus, sharpness, signal to noise ratio, resolution, etc., meet predefined thresholds deemed acceptable for the requested clinical task to be performed); 5) validation of digital characteristics of the medical image (i.e., to ensure that certain digital characteristics, such as compression level, compression loss and artifacts due to compression, bit depth, color space, resolution/pixel density, etc., meet predefined thresholds deemed acceptable for the requested clinical task to be performed); and 6) validation of the physical qualities of the sample captured in the medical image (i.e., to ensure that at the time of capture, the biological sample itself was not degraded or incomplete and thus acceptable for the requested clinical task to be performed).

The measures of suitability of the signal (i.e., the medical image data), for a specific clinical task can be evaluated via one or more approaches. For example, validation of the suitability of a medical image may be based on analysis of the pathophysiological features of the sample in comparison to known pathophysiological features required for a given clinical task. In particular, the system may be configured to process the medical image and identify specific pathophysiological features of a sample, such as certain cell types present and the concentration of said cell types. The system is configured to correlate the identified pathophysiological features of the sample of the medical image with reference data associated with the requested clinical task. For example, the system of the present invention may be configured to access a reference database prepopulated with a plurality of known clinical tasks and known associated pathophysiological features necessary to be present in a given sample in order to perform each of the known clinical tasks. For example, in the case of a user submitting an image of a thin blood film of a patient sample, and further requesting the clinical task of detection of sickle cells in said image, the system is configured to access reference data for the requested clinical task (i.e., the clinical task of detecting sickle cells). The reference data may include, for example, a known request (i.e., the clinical task of detecting sickle cells in a thin blood film sample) and an associated known pathophysiological feature needed to be present in the sample in order to accurately perform the clinical task, which may include, for example a known predefined number/concentration of red blood that must be present in the sample in order to perform the task. Accordingly, upon a positive correlation (i.e., there are a sufficient number of red blood cells present in the patient sample), then the system is able to provide a positive validation, and, upon a negative correlation (i.e., there are an insufficient number of red blood cells present in the patient sample), then the system is able to provide a negative validation.

The system may also provide a direct estimation of clinical suitability using artificial intelligence techniques, including use of artificial neural networks having been trained on the subjective opinion of matter experts. In particular, the system is configured to utilize a neural network architecture design and a corresponding dataset building methodology that permits the training of neural networks to learn human subjective assessments of inputs. The dataset is built from a plurality of pairs of samples (i.e., pairs of images) that have been presented to human experts, in which, for each pair, the human experts have designated one of the pair of images as being generally superior on a subjective scale (i.e., said image has better quality, is more suitable for a clinical task, etc.). In particular, the system makes use of conventional neural networks pre-trained for each of the specific medical data types using a self-supervised methodology.

Upon performing the validation analysis of a given request (i.e., the one or more submitted medical images and associated requested clinical tasks), the system is configured to provide an output indicative of the suitability of the one or more medical images to be used for the specified clinical task, based on the validation analysis. The output can include, for example, a qualitative answer (i.e., a yes/no answer) and/or a quantitative score regarding suitability, which is generally indicative of how successful human experts will be if they used the medical image in question when making a clinical judgment with regards to the specified clinical task.

For example, in the event that the system determines that the request data (i.e., medical image data and/or user's clinical task request) fails to meet a predefined threshold in any of the above described validation stages, the system determines that the input (i.e., the one or more medical images and/or the user request) is unsuitable for clinical use as requested and further alerts the user as to the unsuitability determination. For example, the system may be configured to provide a qualitative yes/no answer, such that, any failure at this stage will cause rejection of the sample, the system will provide a “no” assessment to the user indicating that the medical image is not suitable to be analyzed/used for the requested clinical task, and the request and image will not be processed any further. In the event that the system determines that the request data meets the predefined thresholds in each of the various validation steps described above, then the system will provide a “yes” assessment to the user indicating that the medical image is suitable to be used by qualified human experts to perform the specified clinical task.

In some embodiments, the output may be a value, such as a specific number within a range of 0 and 100 that quantifies the suitability of image to be used for the specified clinical task, in which 0 means the image is least suitable and 100 means the image is most suitable to be used for the specified clinical task. The outputted value (i.e., the number between 0 and 100) can also be used by downstream automated systems for aggregation of results and/or for quality assessment, or during research and development to evaluate incremental progress toward the most optimal image/signal quality.

Accordingly, the system of the present invention is particularly advantageous in that it allows hardware and software manufacturers to update and upgrade their hardware and software as frequently as needed while verifying that the output images still remain suitable for clinical use per scores calculated by the software of the present invention. This in turn makes advances in hardware, sensors and software technology more readily available to help care for patients. As such, the hardware of any given manufacturer does not need to be approved by regulatory bodies, because the software of the present invention is able to check any given hardware's output for clinical suitability. The present invention further allows software and AI developers to use any pathology imaging hardware in a single regulatory approval, by claiming that their product is valid on all “images deemed suitable for human experts” per suitability criteria calculated by the present invention, regardless of their source device. As such, the present invention provides flexibility for hardware-related and software-related digital pathology companies, thereby allowing healthcare providers to be able to change and/or update their technological infrastructure without the worry of regulatory compatibility of pathology hardware and/or software.

Accordingly, the present invention allows for pathology imaging companies to enter the clinical market faster, cheaper, and further encourages technological iteration, while retaining regulatory standards.

### BRIEF DESCRIPTION OF THE DRAWINGS

Features and advantages of the claimed subject matter will be apparent from the following detailed description of embodiments consistent therewith, which description should be considered with reference to the accompanying drawings.

FIG. 1 is a block diagram illustrating one embodiment of an exemplary system for providing automated assessment of suitability of medical data for clinical use consistent with the present disclosure.

FIG. 2 is a block diagram illustrating the medical data assessment system of FIG. 1, including a machine learning module, in greater detail.

FIG. 3 is a block diagram illustrating an exemplary computing system for implementing one or more servers for running the medical data assessment system consistent with the present disclosure.

FIG. 4 shows is a flow diagram generally illustrating the overall process by which the system of the present invention is configured to process input received from a user (including processing of one or more medical images and the user's clinical task request) so as to provide a validation of the request data (i.e., validating various parameters of the inputted medical image data as well as the clinical task request).

FIG. 5 is a block diagram illustrating inputting of reference data (i.e., training data sets) into the machine learning module.

FIG. 6 shows a machine learning module according to certain embodiments of the present disclosure.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

By way of overview, the present invention is directed to a system for providing automated assessment of suitability of medical data for clinical use, including use for the diagnosis of disease and/or planning or evaluation of treatment.

The invention of the present disclosure addresses issues currently faced by medical device manufacturers with regard to regulatory approval of medical devices they create and health care providers with regards to the regulatory compliance of the hardware and software medical devices and their combinations used in patient care. In particular, the present invention is directed to a system for providing automated assessment of the suitability of medical data for clinical use. Such medical data (also referred to as healthcare data) may include, but is not limited to, digital data that may be in the form of image data generally associated with a patient's medical examinations or tests. It should be noted that, while the following description makes reference to the medical data as including medical image(s), the system of the present invention is configured to receive all forms of medical data and is not limited to images. For example, the system of the present invention may be used for assessing the clinical suitability of medical data provided via audio-based files, in addition to image-based files.

As described in greater detail herein, the present invention includes a system for evaluating the suitability of medical images for clinical use, including diagnosis of disease, as well as planning and/or evaluation of treatments. In some embodiments, the medical images may be associated with histologic examinations, which may include one or more images of patient tissue specimens that may be collected and prepared via histological-related techniques. For example, the system is configured to receive one or more images of a histologic examination (i.e., images of one or more histologically prepared tissue samples), such as a peripheral blood smear digitized by a slide scanner. However, it should be noted that the medical images may be associated with certain examinations/tests generally performed via a medical imaging modality and thus may include medical images provided via x-ray imaging, Magnetic Resonance Imaging (MRI), Computed Tomography (CT) imaging, ultrasound (US) imaging, and the like. It should further be noted that, in some embodiments, the medical data may include other forms of data associated with medical- or healthcare-related examinations and tests.

The system is further configured to receive a request from a user associated with a clinical task or use case, essentially requesting that the system determine whether the one or more images are suitable for use in accomplishing such a requested clinical task. For example, a user may request that the system assess whether the one or more images are suitable for use in the clinical task of detection of sickle cells.

In turn, the system is configured to analyze the one or more images, including evaluating the one or more images in different stages and at different predefined levels using a variety of evaluation techniques to determine the suitability of the one or more images for being used for the requested clinical task.

The measures of suitability of the signal (i.e., the medical image data), for a specific clinical task can be evaluated via one or more approaches. For example, validation of the suitability of a medical image may be based on analysis of the pathophysiological features of the sample in comparison to known pathophysiological features required for a given clinical task. In particular, the system may be configured to process the medical image and identify specific pathophysiological features of a sample, such as certain cell types present and the concentration of said cell types. The system is configured to correlate the identified pathophysiological features of the sample of the medical image with reference data associated with the requested clinical task. For example, the system of the present invention may be configured to access a reference database prepopulated with a plurality of known clinical tasks and known associated pathophysiological features necessary to be present in a given sample in order to perform each of the known clinical tasks. For example, in the case of a user submitting an image of a thin blood film of a patient sample, and further requesting the clinical task of detection of sickle cells in said image, the system is configured to access reference data for the requested clinical task (i.e., the clinical task of detecting sickle cells). The reference data may include, for example, a known request (i.e., the clinical task of detecting sickle cells in a thin blood film sample) and an associated known pathophysiological feature needed to be present in the sample in order to accurately perform the clinical task, which may include, for example a known predefined number/concentration of red blood that must be present in the sample in order to perform the task. Accordingly, upon a positive correlation (i.e., there are a sufficient number of red blood cells present in the patient sample), then the system is able to provide a positive validation, and, upon a negative correlation (i.e., there are an insufficient number of red blood cells present in the patient sample), then the system is able to provide a negative validation.

The system may also provide a direct estimation of clinical suitability using artificial intelligence techniques, including using of artificial neural networks having been trained on the subjective opinion of matter experts. In particular, the system is configured to utilize a neural network architecture design and a corresponding dataset building methodology that permits the training of neural networks to learn human subjective assessments of inputs. The dataset is built from a plurality of pairs of samples (i.e., pairs of images) that have been presented to human experts, in which, for each pair, the human experts have designated one of the pair of images as being generally superior on a subjective scale (i.e., said image has better quality, is more suitable for a clinical task, etc.). In particular, the system makes use of conventional neural networks pre-trained for each of the specific medical data types using a self-supervised methodology.

Upon performing the validation analysis of a given request (i.e., the one or more submitted medical images and associated requested clinical tasks), the system is configured to provide an output indicative of the suitability of the one or more medical images to be used for the specified clinical task, based on the validation analysis. The output can include, for example, a qualitative answer (i.e., a yes/no answer) and/or a quantitative score regarding suitability, which is generally indicative of how successful human experts will be if they used the medical image in question when making a clinical judgment in regards to the specified clinical task.

Accordingly, the system of the present invention is particularly advantageous in that it allows hardware and software manufacturers to update and upgrade their hardware and software as frequently as needed while verifying that the output images still remain suitable for clinical use per scores calculated by the software of the present invention. This in turn makes advances in hardware, sensors and software technology more readily available to help care for patients. As such, the hardware of any given manufacturer does not need to be approved by regulatory bodies, because the software of the present invention is able to check any given hardware's output for clinical suitability. The present invention further allows software and AI developers to use any pathology imaging hardware in a single regulatory approval, by claiming that their product is valid on all “images deemed suitable for human experts” per suitability criteria calculated by the present invention, regardless of their source device. As such, the present invention provides flexibility for hardware-related and software-related digital pathology companies, thereby allowing healthcare providers to be able to change and/or update their technological infrastructure without the worry of regulatory compatibility of pathology hardware and/or software.

Accordingly, the present invention allows for pathology imaging companies to enter the clinical market faster, cheaper, and further encourages technological iteration, while retaining regulatory standards.

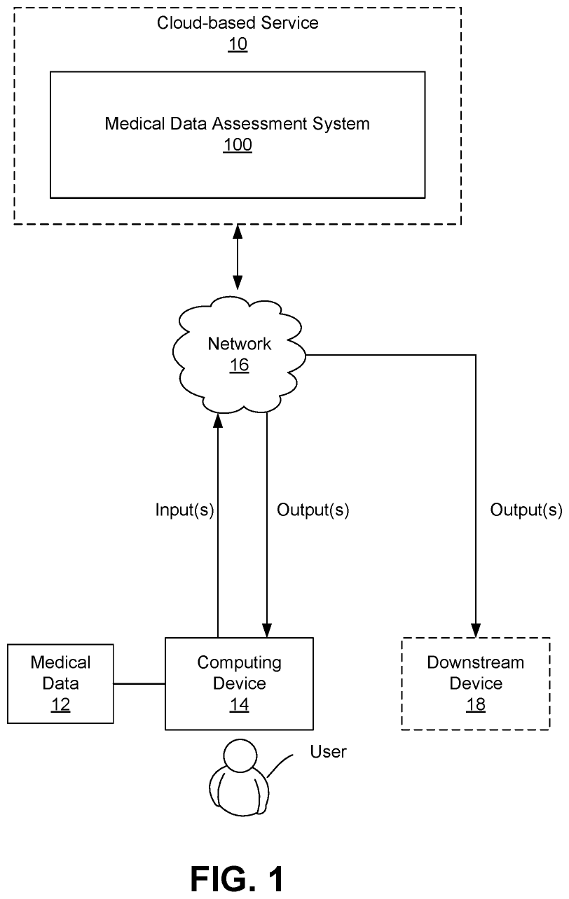

FIG. 1 is a block diagram illustrating one embodiment of an exemplary system for providing automated assessment of suitability of medical data for clinical use consistent with the present disclosure. As shown, the automated medical data assessment system 100 may be embodied on an internet-based computing system/service. For example, as shown, the system 100 may be embodied on a cloud-based service 10, for example. The automated medical data assessment system 100 is configured to communicate and share data with at least a computing device 14 associated with a user. The computing device 14 may be embodied as, without limitation, a computer, a desktop computer, a personal computer (PC), a tablet computer, a laptop computer, a notebook computer, a mobile computing device, a smart phone, a cellular telephone, a handset, a messaging device, a work station, a distributed computing system, a multiprocessor system, a processor-based system, and/or any other computing device configured to store and access data, and/or to execute software and related applications consistent with the present disclosure. In the embodiments described here, the client device 14 is generally embodied as a computer, a desktop computer, a personal computer (PC), a tablet computer, a laptop computer, a notebook computer, and the like.

In some embodiments, the system may also be configured to communicate with a downstream device 18, as described in greater detail herein. The system 100 is configured to communicate and exchange data with the computing device 14 (and the downstream device 18) over a network 16, for example.

The network 16 may represent, for example, a private or non-private local area network (LAN), personal area network (PAN), storage area network (SAN), backbone network, global area network (GAN), wide area network (WAN), or collection of any such computer networks such as an intranet, extranet or the Internet (i.e., a global system of interconnected network upon which various applications or service run including, for example, the World Wide Web). In alternative embodiments, the communication path between the computing device 14 and the system 100 may be, in whole or in part, a wired connection.

The network 16 may be any network that carries data. Non-limiting examples of suitable networks that may be used as network 18 include Wi-Fi wireless data communication technology, the internet, private networks, virtual private networks (VPN), public switch telephone networks (PSTN), integrated services digital networks (ISDN), digital subscriber link networks (DSL), various second generation (2G), third generation (3G), fourth generation (4G), fifth generation (5G), and future generations of cellular-based data communication technologies, Bluetooth radio, Near Field Communication (NFC), the most recently published versions of IEEE 802.11 transmission protocol standards, other networks capable of carrying data, and combinations thereof. In some embodiments, network 16 is chosen from the internet, at least one wireless network, at least one cellular telephone network, and combinations thereof. As such, the network 16 may include any number of additional devices, such as additional computers, routers, and switches, to facilitate communications. In some embodiments, the network 16 may be or include a single network, and in other embodiments the network 16 may be or include a collection of networks.

The 100 is configured to communicate and share data with client devices 14 associated with a user, that may include, for example, a clinician. For example, as previously described herein, this present invention is directed to a software system configured to evaluate the suitability of medical images (among other medical data) for clinical use, including diagnosis of disease and planning or evaluation of treatments. Accordingly, a user, such as a clinician or other medical professional, may utilize the system of the present invention in order to ascertain whether a particular medical image is sufficient (i.e., suitable) for a clinical task, such as performing a diagnostic assessment of content shown in the medical image.

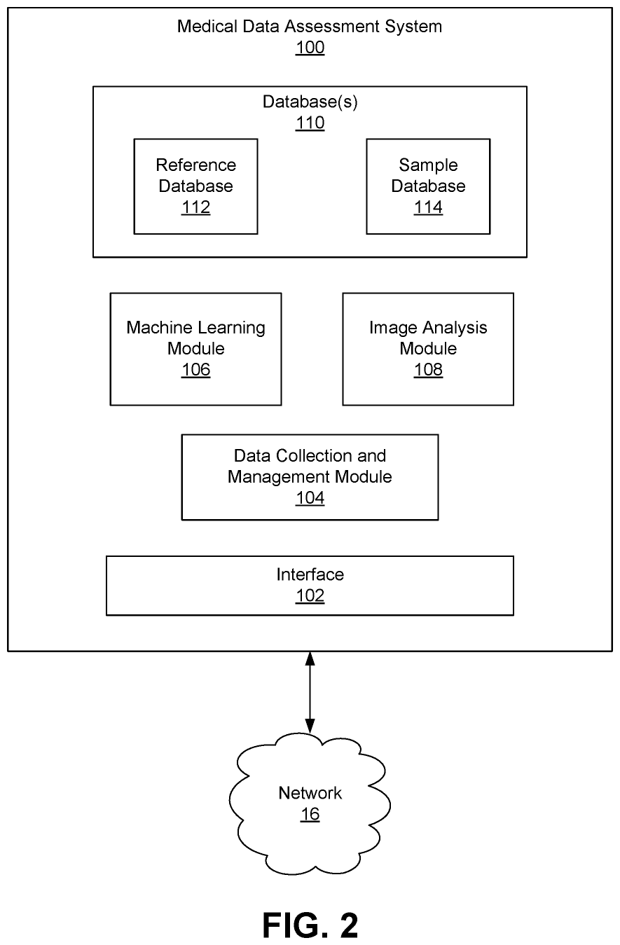

FIG. 2 is a block diagram illustrating the automated medical data assessment system of FIG. 1, including a machine learning module 106, for providing automated assessment of suitability of medical data for clinical use. The system 100 is preferably implemented in a tangible computer system built for implementing the various methods described herein.

As shown, the system 100 may include an interface 102, a data collection and management module 104, a machine learning module 106, an image analysis module 108, and various databases 116 for storage of data, including, but not limited to, a reference database 112 and a sample database 114.

As illustrated, the system 100 may generally be accessed by a user, to initiate methods of the invention and obtain results, via an interface 102, for example. The interface 102 allows for a user to connect with a platform provided via the system and initiate requests for clinical suitability assessments of medical data, specifically allowing for a user to submit one or more medical images to undergo automated analysis and feedback as to whether such medical images are deemed suitable for use in a desired clinical task, as chosen by the user. The system 100 may further include one or more databases 110 with which the machine learning module 106 communicates. In the present example, a reference database 112 includes stored reference data obtained from a plurality of training data sets and a sample database 114 includes stored sample data acquired as a result of evaluations carried out via the system 100 on sample medical images.

Accordingly, the databases 116 may include, for example, training data for training artificial intelligence algorithms and machine learning models, test/validation data for testing trained artificial intelligence and machine learning models, parameters and/or coefficients for trained machine learning models, outputs of machine learning models, and/or any other data that can be used for implementing embodiments of the platform 100.

The system 100 further includes an image analysis module 110 for processing and analyzing incoming medical images to thereby identify characteristics of the medical image (both characteristics of the medical image file as well as characteristics of the content of the medical image) for carrying out the clinical suitability assessments, as will be described in greater detail herein. The data collection and management module 104 may be configured to communicate and exchange data with each of the databases.

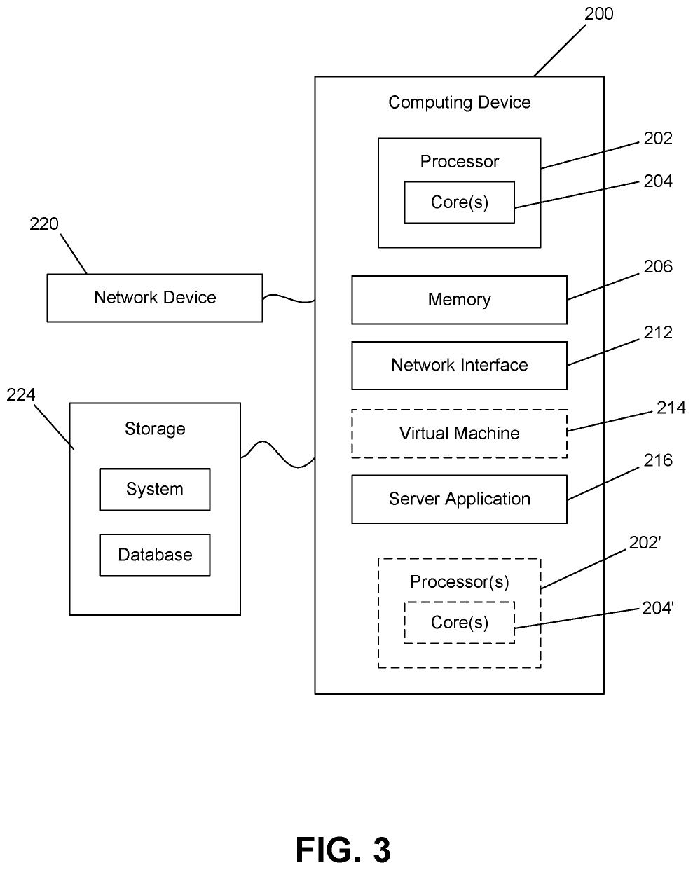

FIG. 3 is a block diagram illustrating an exemplary computing system 200 for implementing one or more servers for running the medical data assessment system 100 consistent with the present disclosure. In the present embodiment, the computing system 200 is configured as a server that is programmed and/or configured to execute one of more of the operations and/or functions for embodiments of the system 100 described herein and to facilitate communication with the user devices via the user interface. The computing system 200 includes one or more non-transitory computer-readable media for storing one or more computer-executable instructions or software for implementing exemplary embodiments. The non-transitory computer-readable media may include, but are not limited to, one or more types of hardware memory, non-transitory tangible media (for example, one or more magnetic storage disks, one or more optical disks, one or more solid state drives), and the like. For example, memory 206 included in the computing system 200 can store computer-readable and computer-executable instructions or software for implementing exemplary embodiments of the components/modules of the platform 100 or portions thereof. The computing system 200 also includes configurable and/or programmable processor 202 and associated core 204, and optionally, one or more additional configurable and/or programmable processor(s) 202′ (e.g., central processing unit, graphical processing unit, etc.) and associated core(s) 204′ (for example, in the case of computer systems having multiple processors/cores), for executing computer-readable and computer-executable instructions or software stored in the memory 206 and other programs for controlling system hardware. Processor 202 and processor(s) 202′ may each be a single core processor or multiple core (204 and 204′) processor.

Virtualization may be employed in the computing system 200 so that infrastructure and resources in the computing device may be shared dynamically. One or more virtual machines 214 may be provided to handle a process running on multiple processors so that the process appears to be using only one computing resource rather than multiple computing resources. Multiple virtual machines may also be used with one processor.

Memory 206 may include a computer system memory or random access memory, such as DRAM, SRAM, EDO RAM, and the like. Memory 206 may include other types of memory as well, or combinations thereof.

The computing system 200 may include or be operatively coupled to one or more data storage devices 224, such as a hard-drive, CD-ROM, mass storage flash drive, or other computer readable media, for storing data and computer-readable instructions and/or software that can be executed by the processing device 202 to implement exemplary embodiments of the components/modules described herein with reference to the platform 100.

The computing system 200 can include a network interface 212 configured to interface via one or more network devices 220 with one or more networks, for example, a Local Area Network (LAN), Wide Area Network (WAN) or the Internet through a variety of connections including, but not limited to, standard telephone lines, LAN or WAN links (for example, 802.11, T1, T3, 56 kb, X.25), broadband connections (for example, ISDN, Frame Relay, ATM), wireless connections (including via cellular base stations), controller area network (CAN), or some combination of any or all of the above. The network interface 212 may include a built-in network adapter, network interface card, PCMCIA network card, card bus network adapter, wireless network adapter, USB network adapter, modem or any other device suitable for interfacing the computing system 200 to any type of network capable of communication and performing the operations described herein. While the system device 200 depicted in block diagram is implemented as a server, exemplary embodiments of the computing system 200 can be any computer system, such as a workstation, desktop computer or other form of computing or telecommunications device that is capable of communication with other devices either by wireless communication or wired communication and that has sufficient processor power and memory capacity to perform the operations described herein.

The computing system 200 may run any server operating system or application 216, such as any of the versions of server applications including any Unix-based server applications, Linux-based server application, any proprietary server applications, or any other server applications capable of running on the computing system 200 and performing the operations described herein. An example of a server application that can run on the computing device includes the Apache server application.

As previously described herein, the system 100 provides automated assessment of the suitability of medical data for clinical use. Such medical data (also referred to as healthcare data) may include, but is not limited to, digital data that may be in the form of image data generally associated with a patient medical examinations or tests.

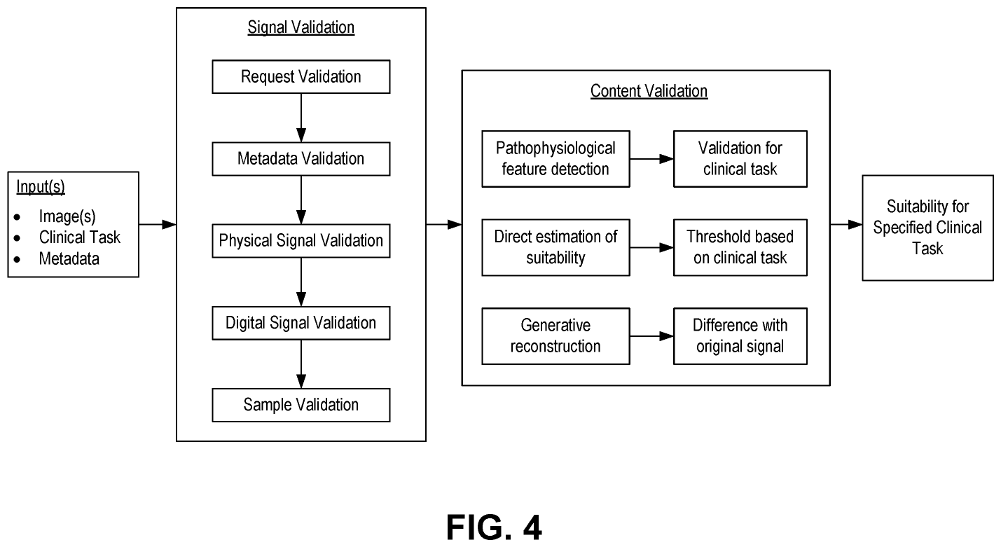

FIG. 4 shows is a flow diagram generally illustrating the overall process by which the system of the present invention is configured to process input received from a user (including processing of one or more medical images and the user's clinical task request) so as to provide a validation of the request data (i.e., validating various parameters of the inputted medical image data as well as the clinical task request).

For example, a user may initiate an assessment by providing, to the system, an assessment request comprising a first input including medical data and a second input including a selected clinical task in which the suitability of use of the medical data for said clinical task is to be assessed. Again, in the embodiments described herein, the medical data is generally digital data, including one or more images. In some embodiments, the medical images may be associated with histologic examinations, which may include one or more images of patient tissue specimens that may be collected and prepared via histological-related techniques. For example, the system is configured to receive one or more images of a histologic examination (i.e., images of one or more histologically prepared tissue samples), such as a peripheral blood smear digitized by a slide scanner. However, it should be noted that the medical images may be associated with certain examinations/tests generally performed via a medical imaging modality and thus may include medical images provided via x-ray imaging, Magnetic Resonance Imaging (MRI), Computed Tomography (CT) imaging, ultrasound (US) imaging, and the like.

The system 100 is further configured to receive a request from a user associated with a clinical task or use case, essentially requesting that the system 100 determine whether the one or more images are suitable for use in accomplishing such a requested clinical task. For example, a user may request that the system assess whether the one or more images are suitable for use in the clinical task of detection of sickle cells.

In turn, the system 100 is configured to analyze the one or more images, including evaluating the one or more images in different stages and at different predefined levels using a variety of evaluation techniques to determine the suitability of the one or more images for being used for the requested clinical task.

For example, the system is configured to process the one or more medical images and the user's clinical task request so as to provide a validation of the request data (i.e., validating various parameters of the inputted medical image data as well as the clinical task request).

Such validation of the request data includes, but is not limited to: 1) validation of the integrity of the medical image (i.e., to ensure that medical image data has not been modified, degraded, or comprised during transmission to the system or during storage thereof); 2) validation of the of the user's request (i.e., verifying that the specifical clinical task request by the user is available for processing by the system); 3) validation of accuracy of metadata associated with the medical image data (i.e., to ensure that the received medical image data matches metadata information); 4) validation of physical characteristics of the medical image (i.e., to ensure that certain physical characteristics, such as optical focus, sharpness, signal to noise ratio, resolution, etc., meet predefined thresholds deemed acceptable for the requested clinical task to be performed); 5) validation of digital characteristics of the medical image (i.e., to ensure that certain digital characteristics, such as compression level, compression loss and artifacts due to compression, bit depth, color space, resolution/pixel density, etc., meet predefined thresholds deemed acceptable for the requested clinical task to be performed); and 6) validation of the physical qualities of the biological sample captured in the medical image (i.e., to ensure that the sample itself was not degraded at the time of capture or was not incomplete or otherwise unsuitable and thus acceptable for the requested clinical task to be performed).

It should be noted that at any of the validation stages previously described herein, the particular thresholds are defined for each clinical task. For example, a clinical task such as the detection of sickle cells can be performed on a very wide range of histological stainings, a wide range of image magnifications, a very wide range of microscope/camera/capture device color balance and color saturation settings, while differentiation of some subclasses of premature white blood cells require very high resolution images, stained only with few acceptable staining methods, with proper white balance. As a result, an image can be suitable for detection of sickle cells while the same image can fail the suitability test for differentiation of immature white blood cells. Accordingly, the particular clinical task is determinative of the particular characteristics and predefined thresholds for such characteristics.

With regard to the validation of the integrity of the medical image, as well as validation of the user's request, cryptographic methods are generally used so as to ensure the integrity of the request and its source is validated. Then the integrity of the medical image is assessed, thereby ensure that the image and other inputs are not degraded or tampered with through the transmission or storage. At this stage, the validity of the request is also assessed by verifying that the evaluation of suitability for the specific clinical task requested by the user is available to the software system processing the request.

More specifically, as part of the product setup, a chain of trust may be established that ensures the system can cryptographically identify the issuer of a signature associated with a request, as well as detect any modifications to or corruptions and degradations of the request or signal that may have occurred in transit, which ensures the system has received a request exactly as issued by the signing party and the request contains a medical image signal as it was provided by the signing party.

After a request to process a medical signal is received as it was intended by the requester, the system needs to validate the information provided about the medical image signal. This validation includes both common sense checks (aka sanity checks) as well as verifications that the provided signal actually matches the claims and specifications provided.

An example of validation of metadata is verifying that the magnification (or pixel density) of an image of a peripheral blood smear matches the claimed magnification in the meta data. Without the use of this invention, these validation are often done automatically by human experts either by following a protocol or merely by relying on intuition and gut feeling. For common sense validation of metadata, standard data validation techniques are used, such as parsing date and time and checking to see if it is within an acceptable range. Additionally, there are signal specific validations that may need to be performed. For example, if there is a request to process an audio recording of a fetal heart, decoding the audio signal and comparing the actual duration of the audio signal with the claimed length in metadata is performed at this stage. In the case of a pathology image, decoding the JPEG files and checking if the width and height of the decoded image matches the metadata provided is done at this stage.

The system 100 is further able to validate physical characteristics of the medical image. At this level, certain properties like optical focus, sharpness, signal to noise ratio, resolution and size of the medical image signal are evaluated. For example, this validation may include checking appropriate illumination (not too dark or too bright) and checking for proper color saturation and white balance. Distortions of the physical samples, like non uniformities of physical specimen, stretching and smearing, wrinkles and tears, can be detected at this stage as well as later stages where the biological context of the image is evaluated. Acceptable thresholds for each measurement is determined based on the particular clinical task specified in the request. At this stage, for each type of measurement and validation, the system has access to predefined tools via the image analysis module 108. These tools are custom made and optimized for each specific measurement type and signal format from the specific source. For example, detection of chromatic aberration in H&E images may be achieved with statistical image processing models that accept the image as well as metadata about magnification and illumination and staining methods as input and generate a scalar value indicating the strength of the chromatic aberration in the image. However, the same task may also be performed by training a deep-neural network based regression model built on top of a foundation modal that is trained specifically in the space pathology samples with similar properties (as described in greater detail herein).

The choice of method for each signal validation task is the core of the system of the present invention. These choices are done by collecting empirical evidence and side-by-side comparison with human experts. For each signal type and new source of signal, a new experiment may be performed to make sure the optimal combination of tools are used to validate the signals.

In scenarios where computational resources or training data availability are not the bottleneck, using deep neural networks will be the practically optimal approach. These neural networks need to be trained on top of domain specific foundation models pre-trained for the specific domain of signals. These foundation models are trained using self-supervised techniques to most accurately represent the space of each particular medical signal type as produced by the relevant technologies and procedures.

An example of these models is a foundation model that represents the space of Romanowsky-stained human peripheral blood smears captured using bright field microscopy in magnifications between 100-1000× by CMOS sensors. After training the encoder half of this model then can be used to train classifiers or regression models that can measure signal quality in this particular space.

Additionally validation of signal at this stage is performed by considering two dimensions: 1) biomedical nature of the signal; and 2) the intended clinical application. For example, to evaluate microscopic images of peripheral blood samples, the system 100 looks up a curated list of properties that need validation from a database. However, the acceptable range of these properties may be dependent on the intended use of the image. In this example, for all microscopic images of peripheral blood magnification is a property that needs to be measured. However, acceptable values for magnification depend on the clinical task included in the request. A magnification of 100× may be considered acceptable for diagnosis of sickle cell disease, while a magnification of 1000× may be required for differentiation of certain leukemias.

The system 100 is further able to validate digital characteristics of the medical image. At this level, the system 100 is checking digital qualities of the medical image signal, including, but not limited to, compression level, compression loss and artifacts due to compression, bit depth, color space, resolution/pixel density, etc. Distortions of the image as a result of digitization, like stitching errors, misalignments, missing segments, stretching and smearing will be detected at this stage as well as later stages where the biological features are evaluated.

The system 100 is further able to validate physical qualities of the sample captured in the medical image. For example, the system 100 is able to detect if an image of a blood smear shows physical damage or staining artifacts. In the case of data reading, the system 100 is able to detect if an EKG signal recording shows the leads were connected in the wrong order to the patient's limbs. In the case of an imaging modality output, the system is able to detect if the CT scan of a patient's chest shows they had a cloth with heavy metallic clasps on, causing a shadow distorting the image. Acceptable thresholds for each measurement is determined based on the particular clinical task specified in the request.

Upon performing the validation analysis of a given request (i.e., the one or more submitted medical images and associated requested clinical tasks), the system is configured to provide an output indicative of the suitability of the one or more medical images to be used for the specified clinical task, based on the validation analysis. The output can include, for example, a qualitative answer (i.e., a yes/no answer) and/or a quantitative score regarding suitability, which is generally indicative of how successful human experts will be if they used the medical image in question when making a clinical judgment in regards to the specified clinical task.

For example, in the event that the system determines that the request data (i.e., medical image data and/or user's clinical task request) fails to meet a predefined threshold in any of the above described validation stages, the system determines that the input (i.e., the one or more medical images and/or the user request) is unsuitable for clinical use as requested and further alerts the user as to the unsuitability determination. For example, the system may be configured to provide a qualitative yes/no answer, such that, any failure at this stage will cause rejection of the sample, the system will provide a “no” assessment to the user indicating that the medical image is not suitable to be analyzed/used for the requested clinical task, and the request and image will not be processed any further. In the event that the system determines that the request data meets the predefined thresholds in each of the various validation steps described above, then the system will provide a “yes” assessment to the user indicating that the medical image is suitable to be used by qualified human experts to perform the specified clinical task.

In some embodiments, the output may be a value, such as a specific number within a range of 0 and 100 that quantifies the suitability of image to be used for the specified clinical task, in which 0 means the image is least suitable and 100 means the image is most suitable to be used for the specified clinical task. The outputted value (i.e., the number between 0 and 100) can be used by downstream automated systems for aggregation of results and/or for quality assessment, or during research and development to evaluate incremental progress toward the most optimal image/signal quality.

The measures of suitability of the signal (i.e., the medical image data), for a specific clinical task can be evaluated via one or more approaches.

For example, validation of the suitability of a medical image may be based on analysis of the pathophysiological features of the sample in comparison to known pathophysiological features required for a given clinical task. In particular, the system may be configured to process the medical image and identify specific pathophysiological features of a sample, such as certain cell types present and the concentration of said cell types. The system is configured to correlate the identified pathophysiological features of the sample of the medical image with reference data associated with the requested clinical task. For example, the system of the present invention may be configured to access a reference database prepopulated with a plurality of known clinical tasks and known associated pathophysiological features necessary to be present in a given sample in order to perform each of the known clinical tasks. For example, in the case of a user submitting an image of a thin blood film of a patient sample, and further requesting the clinical task of detection of sickle cells in said image, the system is configured to access reference data for the requested clinical task (i.e., the clinical task of detecting sickle cells). The reference data may include, for example, a known request (i.e., the clinical task of detecting sickle cells in a thin blood film sample) and an associated known pathophysiological feature needed to be present in the sample in order to accurately perform the clinical task, which may include, for example a known predefined number/concentration of red blood that must be present in the sample in order to perform the task. Accordingly, upon a positive correlation (i.e., there are a sufficient number of red blood cells present in the patient sample), then the system is able to provide a positive validation, and, upon a negative correlation (i.e., there are an insufficient number of red blood cells present in the patient sample), then the system is able to provide a negative validation.

The system may also provide a direct estimation of clinical suitability using artificial intelligence techniques, including using of artificial neural networks having been trained on the subjective opinion of matter experts. In particular, the system is configured to utilize a neural network architecture design and a corresponding dataset building methodology that permits the training of neural networks to learn human subjective assessments of inputs. The dataset is built from a plurality of pairs of samples (i.e., pairs of images) that have been presented to human experts, in which, for each pair, the human experts have designated one of the pair of images as being generally superior on a subjective scale (i.e., said image has better quality, is more suitable for a clinical task, etc.).

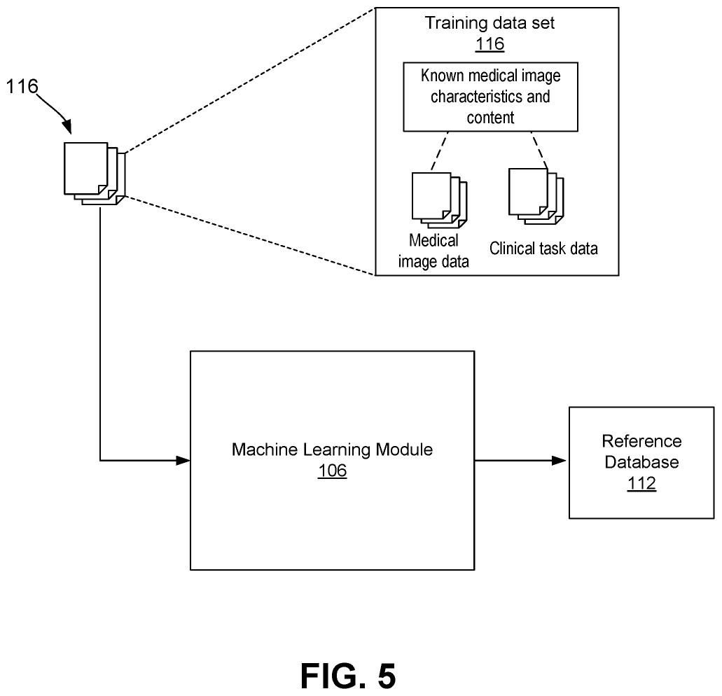

For example, the system 100 may be configured to run a neural network that has been trained using a plurality of training data sets that include qualified reference data. FIG. 5 is a block diagram illustrating inputting of reference data (i.e., training data sets) into the machine learning module. The machine learning techniques of the present invention, and the subsequent analysis and assessment of clinical suitability of medical image(s) based on such techniques, utilize reference data. The reference data may include a plurality of training data sets 116 inputted to a machine learning module 108 of the present invention. For example, each training data set includes reference medical image data, which may include, for example, medical images that include known medical image characteristics and content (i.e., known physical and digital characteristics of the medical image signal as well as pathophysiological characteristics of the content of the medical image). Each training data set further includes known clinical tasks associated with the known medical image characteristics and content. Each known given clinical task has a number of specific medical image characteristics and content associated therewith as well as predefined acceptable thresholds for each (which are determinative of suitability for user in carrying out the specific clinical task).

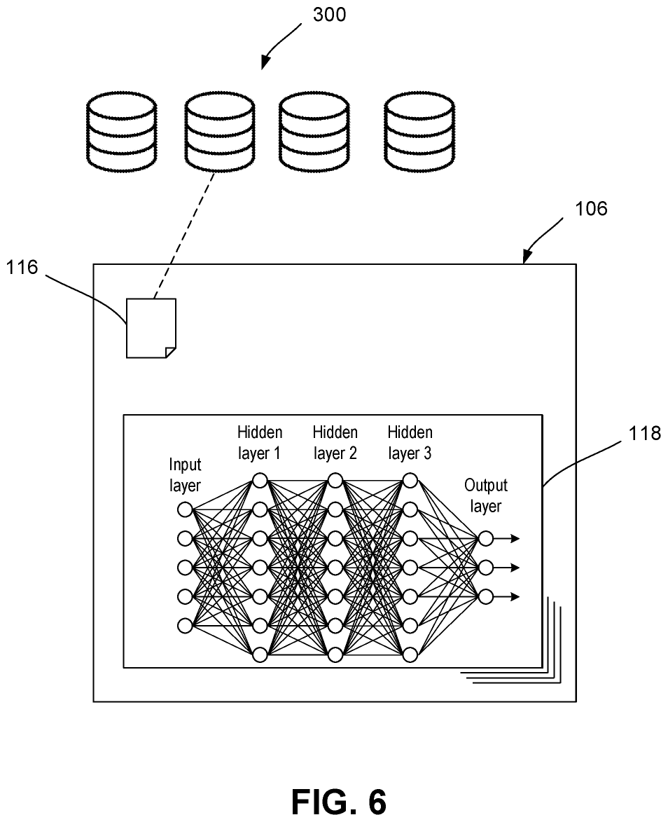

FIG. 6 shows a machine learning module 108 according to certain embodiments of the present disclosure. The machine learning module 108 accesses reference data from the one or more training data sets 116 provided by any known source 300. The source 300 may include, for example, a laboratory-specific repository of reference data collected for purposes of machine learning training. Additionally, or alternatively, the source 300 may include publicly available registries and databases and/or subscription-based data sources. As described herein, the dataset may be built from a plurality of pairs of samples (i.e., pairs of images) that have been presented to human experts, in which, for each pair, the human experts have designated one of the pair of images as being generally superior on a subjective scale (i.e., said image has better quality, is more suitable for a clinical task, etc.). In preferred embodiments, the plurality of training data sets 116 feed into the machine learning module 108. The machine learning module 108 may include, but is not limited to, a neural network, a random forest, a support vector machine, a Bayesian classifier, a Hidden Markov model, an independent component analysis method, and a clustering method.

For example, the machine learning module 108 an autonomous machine learning system that associates the condition data with the reference ultrasound image data. For example, the machine learning system may include a deep learning neural network that includes an input layer, a plurality of hidden layers, and an output layer. The autonomous machine learning system may represent the training data set using a plurality of features, wherein each feature comprises a feature vector. For example, the autonomous machine learning system may include a convolutional neural network (CNN). In the depicted embodiment, the machine learning system 108 includes a neural network 118.

The machine learning system 108 discovers associations in data from the training data sets. In particular, the machine learning module 108 processes and associates the reference medical image data and clinical task data with one another, thereby establishing reference data in which known characteristics of physical and digital properties of medical images and content within are associated with known requirements associated with certain clinical tasks. The reference data is stored within the reference database 112, for example, and available during subsequent processing of sample medical images undergoing suitability assessments.

The system may also provide evaluation of suitability through reconstruction. For example, one measure of signal quality is calculated by sending the input signal through an auto-encoder specifically trained to represent the signals in the particular biological and technological space of the sample, and comparing the output of the auto-encoder with the original signal. The auto-encoders used for this purpose are only trained with signals suitable for the target task and the training parameters are set to generate the highest quality signals. The differences between an incoming signal and the output generated by the auto-encoder is indicative of deviation from the features expected in a signal best suitable for the target task.

With regard to the direct estimation of clinical suitability using artificial intelligence techniques, including using of artificial neural networks having been trained on the subjective opinion of matter experts, it is critical that the trained datasets contains samples that satisfy the following properties: 1) there are samples covering the upper and lower bounds of the applicable ranges; and 2) the majority of samples are concentrated in the region of transition where judgment between samples becomes completely impossible by human experts (where performance of human experts is at chance level).

To use this dataset most efficiently, the present system provides an architecture that uses a combination of existing technologies in a unique way, as noted below:

1. We train conventional neural networks (for example convolutional neural networks in case of images) using a self-supervised methodology (for example using autoencoders+image augmentation).
2. We disassemble the model trained in step 1 above, and take the encoder section to step 3.
3. On top of this decoder we train a classifier or a regression model. For example, in the case of the regression models, a dense multilayer perceptron is added after the decoder that collapses the output of the decoder into a single continuous value with a linear activation the output of which is retrained using normalization.
4. We embed the network from step 3 (encoder+MLP) in a recurrent layer. This allows using pairs of inputs in a sequential manner which not only cuts the memory requirement for training the model in half, it also doubles the training rate by using the same weights to process both inputs.
5. We connect the outputs of the network from step 3 (encoder+MLP) through the recurrent layer to a dense layer which collapses the two inputs into a single node outputting a single quantitative value with a sigmoid activation function or similar activation that allows for training the entire network on the binary annotations of the dataset.
6. After training loss reaches the expected asymptote, the trained network is disassembled one more time and the network from step 3 (encoder+MLP) is extracted.
7. The output of such network is a quantitative measure of the subjective judgements of the human experts. In cases that a yes no answer is desired a short trial with a very small data set can provide the statistical power to choose a threshold on the output.

It should be noted that the system further provides safeguards. Due to the nature of this problem, it is critical that the system errs on the side of caution and rejects the suspicious samples at the expense of being too strict rather than allowing any unsuitable images through to later diagnostic stages. As a result there are multiple layers of safety built into the system.

There are safety measures provided at the level of auto-encoder. The auto-encoders that are used at the “reconstruction state” are trained with these rules in mind: 1) The datasets used for training only contain the highest quality images; and 2) training is partially biased toward faithful reproduction of features that are important to human experts. For example, although it is completely possible for a machine (and human experts if trained) to differentiate between eosinophils from neutrophils in high resolution but grayscale images of blood smears. The vast majority of human experts would claim that throughout their formal clinical training they have learned to depend on the distinct orange-red color of eosinophilic granules to differentiate cosinophils from neutrophils. Thus the system gives priority/weight to more accurate representation and reproduction of such features. This is achieved both by composition of the training dataset and by manual composition of loss function.

The system further provides for general oversight, in the form of semi-annual human expert review and update on the biological feature set and diagnostic requirements. Additionally, the system allows for performance evaluation in the form of continuous automated performance evaluation and/or annual expert performance evaluation.

The system of the present invention is particularly advantageous in that it allows hardware and software manufacturers to update and upgrade their hardware and software as frequently as needed while verifying that the output images still remain suitable for clinical use per scores calculated by the software of the present invention. This in turn makes advances in hardware, sensors and software technology more readily available to help care for patients. As such, the hardware of any given manufacturer does not need to be approved by regulatory bodies, because the software of the present invention is able to check any given hardware's output for clinical suitability. The present invention further allows software and AI developers to use any pathology imaging hardware in a single regulatory approval, by claiming that their product is valid on all “images deemed suitable for human experts” per suitability criteria calculated by the present invention, regardless of their source device. As such, the present invention provides flexibility for hardware-related and software-related digital pathology companies, thereby allowing healthcare providers to be able to change and/or update their technological infrastructure without the worry of regulatory compatibility of pathology hardware and/or software.

Accordingly, the present invention allows for pathology imaging companies to enter the clinical market faster, cheaper, and further encourages technological iteration, while retaining regulatory standards.

As used in any embodiment herein, the term “module” may refer to software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices. “Circuitry”, as used in any embodiment herein, may comprise, for example, singly or in any combination, hardwired circuitry, programmable circuitry such as computer processors comprising one or more individual instruction processing cores, state machine circuitry, and/or firmware that stores instructions executed by programmable circuitry. The modules may, collectively or individually, be embodied as circuitry that forms part of a larger system, for example, an integrated circuit (IC), system on-chip (SoC), desktop computers, laptop computers, tablet computers, servers, smartphones, etc.

Any of the operations described herein may be implemented in a system that includes one or more storage mediums having stored thereon, individually or in combination, instructions that when executed by one or more processors perform the methods. Here, the processor may include, for example, a server CPU, a mobile device CPU, and/or other programmable circuitry.

Also, it is intended that operations described herein may be distributed across a plurality of physical devices, such as processing structures at more than one different physical location. The storage medium may include any type of tangible medium, for example, any type of disk including hard disks, floppy disks, optical disks, compact disk read-only memories (CD-ROMs), compact disk rewritables (CD-RWs), and magneto-optical disks, semiconductor devices such as read-only memories (ROMs), random access memories (RAMs) such as dynamic and static RAMs, erasable programmable read-only memories (EPROMs), electrically erasable programmable read-only memories (EEPROMs), flash memories, Solid State Disks (SSDs), magnetic or optical cards, or any type of media suitable for storing electronic instructions. Other embodiments may be implemented as software modules executed by a programmable control device. The storage medium may be non-transitory.

As described herein, various embodiments may be implemented using hardware elements, software elements, or any combination thereof. Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The term “non-transitory” is to be understood to remove only propagating transitory signals per se from the claim scope and does not relinquish rights to all standard computer-readable media that are not only propagating transitory signals per se. Stated another way, the meaning of the term “non-transitory computer-readable medium” and “non-transitory computer-readable storage medium” should be construed to exclude only those types of transitory computer-readable media which were found in In Re Nuijten to fall outside the scope of patentable subject matter under 35 U.S.C. § 101.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### INCORPORATION BY REFERENCE

References and citations to other documents, such as patents, patent applications, patent publications, journals, books, papers, web contents, have been made throughout this disclosure. All such documents are hereby incorporated herein by reference in their entirety for all purposes.

### EQUIVALENTS

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A system for providing assessment of clinical suitability of medical data, the system comprising:
a computing system configured to communicate with one or more user-associated or software-agent-associated computing devices over a network, the computing system comprising a hardware processor coupled to non-transitory, computer-readable memory containing instructions executable by the processor to cause the computing system to:
receive an assessment request from a user or software-agent, the assessment request comprising a first input including medical data and a second input including a selected clinical task in which the suitability of use of the medical data for said clinical task is to be assessed;
analyze the medical data to at least determine validity of the medical data, said analysis comprising performing one or more determinations of validity of characteristics of the medical data based, at least in part, on processing of the medical data to identify one or more characteristics of the medical data and correlating the one or more characteristics from the processed data with known reference data; and
output an indication of clinical suitability of the medical data for use in the selected clinical task based, at least in part, on the outcome of the validation analysis.

2. The system of claim 1, wherein the first input includes a digital signal comprising data associated with a patient's medical- or healthcare-related examination or test.

3. The system of claim 2, wherein the first input includes a medical image.

4. The system of claim 3, wherein the medical image is associated with an examination or test performed via a medical imaging modality selected from the group consisting of x-ray imaging, Magnetic Resonance Imaging (MRI), Computed Tomography (CT) imaging, and ultrasound (US) imaging.

5. The system of claim 3, wherein the image is associated with a histological examination.

6. The system of claim 5, wherein the image comprises a histologically-prepared patient tissue and/or fluid specimen.

7. The system of claim 2, wherein the one or more determinations of validity of characteristics of the medical image comprises at least one of: validation of the integrity of the medical image; validation of accuracy of metadata associated with the medical image; validation of physical characteristics of the medical image; validation of digital characteristics of the medical image; and validation of the physical qualities of a tissue and/or fluid sample captured in the medical image.

8. The system of claim 7, wherein validation of the integrity of the medical image comprises analyzing medical image data to determine whether the medical image has been modified, degraded, or compromised during transmission to the system or during storage thereof.

9. The system of claim 7, wherein validation of accuracy of metadata associated with the medical image comprises analyzing medical image data to determine whether received medical image data matches metadata information associated with the source medical image.

10. The system of claim 7, wherein validation of physical characteristics of the medical image comprises:
processing the medical image to identify physical characteristics of the medical image selected from the group consisting of optical focus, sharpness, signal to noise ratio, and resolution; and
determining whether said identified physical characteristics meet predefined thresholds associated with the known reference data and deemed acceptable for the requested clinical task to be performed.

11. The system of claim 7, wherein validation of digital characteristics of the medical image comprises:
processing the medical image to identify digital characteristics of the medical image selected from the group consisting of compression level, compression loss and artifacts due to compression, bit depth, color space, and resolution and/or pixel density; and
determining whether said identified digital characteristics meet predefined thresholds associated with the known reference data and deemed acceptable for the requested clinical task to be performed.

12. The system of claim 7, wherein validation of the physical qualities of the tissue and/or fluid sample captured in the medical image comprises:
processing the medical image to identify physical qualities of the tissue and/or fluid sample; and
determining whether said identified physical qualities meet predefined thresholds associated with the known reference data and deemed acceptable for the requested clinical task to be performed.

13. The system of claim 3, wherein the computing system is configured to run a neural network, wherein the neural network has been trained using a plurality of training data sets, each training data set comprises reference medical images associated with known clinical tasks and known characteristics of medical images and content therein associated with the known clinical tasks, and each training data set has been trained based, at least in part, on human subjective assessments of training data set inputs.

14. The system of claim 13, wherein the analysis of at least the medical image for clinical suitability assessments comprises correlating the one or more characteristics from the processed data of the medical image with known characteristics of medical images and content therein and known clinical tasks associated therewith.

15. The system of claim 13, wherein the computing system comprises a machine learning system selected from the group consisting of a neural network, a random forest, a support vector machine, a Bayesian classifier, a Hidden Markov model, an independent component analysis method, and a clustering method.

16. The system of claim 13, wherein the computing system comprises an autonomous machine learning system that associates the known clinical tasks with the known reference data.

17. The system of claim 16, wherein the machine learning system comprises a deep learning neural network that includes an input layer, a plurality of hidden layers, and an output layer.

18. The system of claim 17, wherein the autonomous machine learning system represents the training data set using a plurality of features, wherein each feature comprises a feature vector.

19. The system of claim 16, wherein the autonomous machine learning system comprises a convolutional neural network (CNN) trained using a self-supervised methodology.

20. The system of claim 3, wherein the known reference data comprises a plurality of known clinical tasks and known associated pathophysiological features necessary to be present in a given sample in order to perform each of the known clinical tasks.

21. The system of claim 1, wherein the indication of clinical suitability comprises at least one of a qualitative indication and a quantitative indication.

22. The system of claim 21, wherein a qualitative indication comprises either a positive answer indicating that the medical data is suitable for use in the clinical task or a negative answer indicating that the medical data is unsuitable for use in the clinical task.

23. The system of claim 21, wherein the quantitative indication comprises a suitability score having a value within a defined range indicative of the suitability of the medical data use in the clinical task.

24. The system of claim 23, wherein the suitability score falls within a range of 0 and 100, wherein 0 indicates that the medical data is least suitable for use in the clinical task and 100 indicates that the medical data is most suitable for use in the clinical task.
